(19) United States
(12) Patent Application Publication
(10) Pub. No.: US 2007/0259002 A1
(43) Pub. Date: Nov. 8, 2007

(54) Botulinum Toxin Therapy of Heart Rhythm Disorders

(76) Inventor: John Batchelor, Berkshire (GB)

Correspondence Address: JACOBSON HOLMAN PLLC, 400 SEVENTH STREET N.W., SUITE 600, WASHINGTON, DC, 20004, US

(21) Appl. No.: 11/666,639

(22) PCT Filed: Oct. 28, 2005
(86) PCT No.: PCT/GB05/04182

§ 371 Date: Apr. 30, 2007

(30) Foreign Application Priority Data
Oct. 29, 2004 (GB) 0424048.7

(51) Int. Cl. A61K 39/08; A61P 9/06

(52) U.S. Cl. 424/239.1

(57) ABSTRACT

The instant invention relates to the use of a pre-synaptic neuromuscular blocking substance for preparing a medicament intended to treat or prevent heart fibrillation disorders. Preferably, the pre-synaptic neuromuscular blocking substance will be a botulinum toxin. Preferably also, the heart fibrillation disorder to be treated or prevented will be atrial fibrillation.

The instant invention relates to the use of a pre-synaptic neuromuscular blocking substance for preparing a medicament intended to treat or prevent heart fibrillation disorders.

Among pre-synaptic neuromuscular blocking substances can be mentioned in first instance botulinum toxins. Botulinum toxin is the most lethal natural biological agent known to man. About 50 picograms of a commercially available botulinum toxin type A (purified neurotoxin complex) correspond to the LD₅₀ in mice. However, the same toxin has been used at tiny doses for therapeutic purposes in man since the 1980s. To date, it is believed to treat a number of disorders among which can be mentioned the following: blepharospasm, hemifacial spasm, cervical dystonia, cerebral palsy, post-stroke spasticity of the arm or leg, hyperhidrosis (e.g. palmar hyperhidrosis, axillar hyperhidrosis, Frey's syndrome or gustatory sweating), wrinkles (e.g. glabellar lines), urinary retention, urinary incontinence, benign prostatic hyperplasia, anal fissure, skin wounds, type 2 diabetes, articular pathologies, acne and many others. Other botulinum toxins share the same therapeutic properties of botulinum toxin type A (e.g. botulinum toxins of types B, C₁, D, E, F and G); however, only botulinum toxin type A and botulinum toxin type B are currently marketed. Botulinum toxin type A is mainly commercially available from Ipsen (Dysport®, Ipsen Limited, Slough, UK) and Allergan (BOTOX®, Allergan Inc., Irvine, Calif., USA) whereas botulinum toxin type B is sold by Elan (Myobloc®/Neurobloc®, Solstice Neurosciences Inc., San Diego, Calif., USA).

PCT patent application WO 03/084567 (or U.S. Pat. No. 6,767,544) teaches the use of botulinum toxin to treat a cardiovascular disorder by administering an effective amount of a botulinum toxin directly to a blood vessel of a patient (notably to prevent restenosis). In this patent application, “cardiovascular” is defined as “pertaining to blood vessels, for example, blood vessels of the heart”.

The Applicant has now surprisingly found that a pre-synaptic neuromuscular blocking substance, and in particular a botulinum toxin, could also be used for the treatment or prevention of heart rhythm disorders, notably heart fibrillation disorders.

By pre-synaptic neuromuscular blocking substance should be understood in the present application a substance that prevents and/or inhibits transmission of the chemical messages and signals involved in pre-synaptic neuromuscular activity. Examples of such pre-synaptic neuromuscular blocking substances are substances that inhibit acetylcholine (ACh) synthesis or release; those include notably biological toxins (such as botulinum neurotoxins and bungarotoxins but also any synthetic analogue thereof having the same activity like recombinantly engineered botulinum toxins) and chemicals (such as hemicholinium or triethylcholine which inhibit ACh synthesis, aminoglycoside antibiotics which inhibit ACh release or tubocurarine and similar compounds). Preferred pre-synaptic neuromuscular blocking substances according to this invention will be botulinum neurotoxins and bungarotoxins (α-bungarotoxin being preferred among the bungarotoxins).

By botulinum neurotoxins (or botulinum toxins) is meant in the present application botulinum neurotoxin complexes (whether of type A, B, C, D, E, F or G), high purity botulinum neurotoxins (whether of type A, B, C, D, E, F or G) as well as any recombinantly engineered botulinum toxin having similar or better properties than the naturally occurring botulinum toxins or the purified forms thereof. Botulinum toxin type A includes all types of botulinum toxin type A, including A₁, A₂ and A₃, botulinum toxin type C includes all types of botulinum toxin type C, including C₁ or C₂; the same applies mutatis mutandis to the other serotypes of toxins.

By botulinum neurotoxin complex (whether of type A, B, C, D, E, F or G) should be understood in the present application a botulinum neurotoxin (whether of type A, B, C, D, E, F or G) associated with at least another non-toxic protein.

By high purity botulinum neurotoxin (whether of type A, B, C, D, E, F or G) is meant, in the present application, botulinum neurotoxin (whether of type A, B, C, D, E, F or G) outside from complexes including at least another protein. In other words, a high purity botulinum neurotoxin (type A, B, C, D, E, F or G) does not contain significant quantities of any other Clostridium spp derived protein than botulinum neurotoxin (type A, B, C, D, E, F or G).

Preferably, the pre-synaptic neuromuscular blocking substance will be a botulinum toxin. More preferably, the botulinum toxin will be selected from the group consisting of botulinum toxin type A, botulinum toxin type B and botulinum toxin type F. Even more preferably, the botulinum toxin will be selected from the group consisting of botulinum toxin type A and botulinum toxin type B. In particular, the botulinum toxin will be botulinum toxin type A.

According to the instant invention, the heart rhythm disorders are preferably heart fibrillation disorders. Heart fibrillation disorders are preferably selected from the group consisting of atrial fibrillation and ventricular fibrillation.

According to a first variant of the invention, the heart fibrillation disorder intended to be treated or prevented is atrial fibrillation.

Atrial fibrillation can manifest several ways. Older people typically get chronic atrial fibrillation. Excess adrenaline causes adrenergic atrial fibrillation. Neurogenic atrial fibrillation stems from an imbalance in the nervous system's regulation of the heart. Men between 30 and 50 are subject to vagal atrial fibrillation after a meal or at rest. Young people can be affected by lone or primary atrial fibrillation, which presents no identifiable cause; paroxysmal atrial fibrillation, which causes intermittent attacks of variable length; and rare familial atrial fibrillation. Atrial fibrillation may also be associated with other cardiac muscles or valvula disorders such as cardiomyopathy, cardiac insufficiency, ischemia and/or infarction disorders (e.g. aortic, tricuspid, pulmonary or mitral valvula stenosis) or insufficiency or conduction disorders.

According to a second variant of the invention, the heart fibrillation disorder intended to be treated or prevented is ventricular fibrillation.

The administration locus for the pre-synaptic neuromuscular blocking substance should be the location of the source of the irregular rhythm. In order to find said source, one could for example perform an electrophysiological study.

According to one variant of the invention, the pre-synaptic neuromuscular blocking substance is used to treat the fibrillation disorder. In such case, a pre-synaptic neuromuscular blocking substance with short onset of action like voltage-gated sodium channel blockers will be preferred.

According to another variant of the invention, the pre-synaptic neuromuscular blocking substance is used to prevent recurrence of the fibrillation disorder. In such case, a pre-synaptic neuromuscular blocking substance with a prolonged duration of action (e.g. botulinum toxin type A or botulinum toxin type F, and preferably botulinum toxin type A) will be preferred.

The dose of pre-synaptic neuromuscular blocking substance which shall be needed for the treatment of the disorders mentioned above varies depending on the disorder to be treated, administration mode, age and body weight of the patient to be treated and health state of the latter, and it is the treating physician or veterinary that will eventually make the decision. Such a quantity determined by the treating physician or veterinarian is called here “therapeutically efficient quantity”.

For botulinum neurotoxin complex (type A, B, C, D, E, F or Q) or high purity botulinum neurotoxin (type A, B, C, D, E, F or G), this therapeutically efficient dose is often expressed as a function of the corresponding LD₅₀. By LD₅₀ should be understood in the present application the median intraperitoneal dose in mice injected with botulinum neurotoxin complex (type A, B, C, D, E, F or G) or high purity botulinum neurotoxin (type A, B, C, D, E, F or G) that causes death of half of said mice within 96 hours (see the “Mouse toxicity assay” protocol under “ANALYTICAL METHODS”).

However, it is believed that, for botulinum toxin type A, this effective amount could be about 10 to 500 LD₅₀ units and preferably about 50 to 250 LD₅₀ units:

The approximate effective amounts for the other pre-synaptic neuromuscular blocking substances can easily be deduced by one skilled in the art (by a conversion based on his knowledge of the respective activities of said substances).

Of course, the pre-synaptic neuromuscular blocking substance treatment according to the instant invention may be combined with the other current treatments of heart rhythm disorders (such as pacemaker, anticoagulants or other common medications for the treatment of heart rhythm disorders).

The pre-synaptic neuromuscular blocking substance treatment may optionally be associated with an analgesic treatment (e.g. by administration of an analgesic substance like morphine) prior to, at the same time or shortly after the toxin is administered.

The term “about” refers to an interval around the considered value. As used in this patent application, “about X” means an interval from X minus 10% of X to X plus 10% of X, and preferably an interval from X minus 5% of X to X plus 5% of X and about “about X to Y” means an interval from X minus 10% of (X+Y) to Y plus 10% of (X+Y), and preferably an interval from X minus 5% of (X+Y) to Y plus 5% of (X+Y).

Unless they are defined differently, all the technical and scientific terms used here have the same meaning as that usually understood by an ordinary specialist in the field to which this invention belongs. Similarly, all publications, patent applications, all patents and all other references mentioned here are incorporated by way of reference.

The following example is presented to illustrate the above and must in no case be considered as a limit to the scope of the invention.

### EXAMPLE

### Treatment of Atrial Fibrillation

A male patient in his sixties has had several episodes of atrial fibrillation. An electrophysiological study is performed to determine the location of the source of the irregular rhythm. Injection of 80 LD₅₀ units of a botulinum toxin type A preparation (e.g. Dysport® from Ipsen Ltd, Slough, UK—the product being used following the reconstitution protocol of the manufacturer) into the key trigger points found for this patient is carried out to correct the heart rhythm disorder. As a result, the arrhythmic episodes were eliminated for a period of at least three months.

### ANALYTICAL METHODS

### Mouse Toxicity Assay

A mouse toxicity assay can be used to measure the toxicity of botulinum neurotoxin complex (type A, B, C, D, E, F or G) or high purity botulinum neurotoxin (type A, B, C, D, E, F or G). In the assay, a standard diluent will be used to prepare a range of dilutions at or about the estimated LD₅₀ value. The range and scale of dilutions is arranged so as to establish an accurate LD₅₀ value.

Mice are injected intraperitoneally with a known and standardised volume of diluted toxin. After 96 hours, the number of deaths and survivors in each dilution group will be recorded. The LD₅₀ value is the median dose which kills half of the injected animals within 96 hours.

1. A use of botulinum neurotoxins or synthetic analogues of botulinum neurotoxins, for preparing a medicament intended to treat or prevent atrial fibrillation disorders selected from the group consisting of adrenergic atrial fibrillation, vagal atrial fibrillation, lone or primary atrial fibrillation, paroxysmal atrial fibrillation, familial atrial fibrillation.

2. A use of botulinum neurotoxins or synthetic analogues of botulinum for preparing a medicament intended to prevent recurrence of the fibrillation disorder.

3. The use according to claim 2, characterised in that the botulinum neurotoxins or synthetic analogues of botulinum neurotoxins is a substance with a prolonged duration of action.

4. The use according to claim 1, characterised in that the botulinum neurotoxin is selected from the group consisting of botulinum toxin type A, botulinum toxin type B and botulinum toxin type F.

5. The use according to claim 1, characterised in that the botulinum neurotoxin is selected from the group consisting of botulinum toxin type A and botulinum toxin type B.

6. The use according to claim 1, characterised in that the botulinum neurotoxin is botulinum toxin type A.

7. The use according to claim 1, characterised in that the botulinum neurotoxins or synthetic analogues of botulinum neurotoxins is associated with an analgesic substance.
